(19) United States
(12) Patent Application Publication
(10) Pub. No.: US 2012/0220896 A1
(43) Pub. Date: Aug. 30, 2012

(54) GUIDEWIRE

(75) Inventors: Kazumi MATSUO, Tajimi (JP); Kouji SUGIURA, Kasugai (JP); Fuminori SUGIYAMA, Aichi (JP)

(73) Assignee: ASAHI INTECC CO., LTD., Nagoya-shi (JP)

(21) Appl. No.: 13/345,269

(22) Filed: Jan. 6, 2012

(30) Foreign Application Priority Data
Feb. 28, 2011 (JP) 2011-41430

(51) Int. Cl. A61M 25/09

(52) U.S. Cl. 600/585

(57) ABSTRACT

A guidewire includes a core shaft having a tip portion and a rear end portion, and a hollow stranded-wire body formed by twisting a plurality of strands. The core shaft includes a small-diameter portion including the tip portion and extending over a predetermined area from the tip portion toward the rear end portion, and a large-diameter portion located nearer to the rear end portion than the small-diameter portion and extending over a predetermined area in the direction from the tip portion toward the rear end portion. The small-diameter portion is inserted into the hollow stranded-wire body and covered with the hollow stranded-wire body to partially constitute an outer circumferential surface of the guide wire.

### CROSS REFERENCE TO RELATED APPLICATIONS

This application is based on Japanese Patent Application No. 2011-41430 filed with the Japan Patent Office on Feb. 28, 2011, the entire content of which is hereby incorporated by reference.

### TECHNICAL FIELD

The present invention relates to a guidewire.

### BACKGROUND ART

In the related art, a guidewire is known as a medical device used for percutaneous transluminal coronary angioplasty (hereinafter simply referred to as “PTCA”). The guidewire is used to guide a device such as a balloon and a stent to a lesion site.

Such a guidewire is disclosed in, for example, Japanese Patent No. 2902076. This guidewire includes a core shaft and a coiled body. The core shaft has a tip portion with a small diameter. The core shaft has a tapered shape with its diameter decreasing from a rear end portion toward the tip portion thereof. The coiled body is wound around the tip portion of the core shaft. The core shaft and the coiled body are brazed to each other at a predetermined position. The tip portion of the core shaft of this guidewire corresponds to a distal portion of the guidewire. The rear end portion of the core shaft corresponds to a proximal portion of the guidewire. The distal portion of the guidewire reaches a lesion site after being inserted into the body. The proximal portion of the guidewire is manipulated by an operator such as a physician.

In the guidewire in the related art described in Japanese Patent No. 2902076, the core shaft has the tip portion with a small diameter. Therefore, this guidewire has a high flexibility, so that the tip portion of the core shaft may have less chance to damage the inner wall of the vessel.

### SUMMARY OF INVENTION

In the guidewire in the related art described in Japanese Patent No. 2902076, the coiled body is formed by spirally winding a single strand around the tip portion of the core shaft, with the core shaft being the central axis thereof. Therefore, the following problem occurs. Rotation of the proximal portion of the guidewire applies a torsional force to, and thus easily twists, the coiled body. Therefore, torque generated at the proximal portion of the guidewire is less likely to be transmitted to the distal portion of the guidewire. As a result, when a lesion site is hardened or includes a severe stenosis or an occlusion, the distal portion of the guidewire may hardly pass through the lesion site.

The present inventors have conducted intensive studies to solve the problem. They have found, as a result, that the use of a wire rope instead of the coiled body facilitates transmission of the torque generated at the proximal portion of the guidewire to the distal portion of the guidewire. The present inventors have completed the guidewire according to the present invention on the basis of the above finding.

A guidewire according to the present invention includes: a core shaft having a tip portion and a rear end portion; and a hollow stranded-wire body formed by twisting a plurality of strands, wherein the core shaft includes: a small-diameter portion including the tip portion and extending over a predetermined area from the tip portion toward the rear end portion; and a large-diameter portion located nearer to the rear end portion than the small-diameter portion and extending over a predetermined area in the direction from the tip portion toward the rear end portion, and the small-diameter portion is inserted into the hollow stranded-wire body and covered with the hollow stranded-wire body to partially constitute an outer circumferential surface of the guide wire.

In the guidewire according to the present invention, desirably, the large-diameter portion has substantially the same maximum diameter as the hollow stranded-wire body.

In the guidewire according to the present invention, desirably, a stepped portion is formed on the outer circumferential surface of the hollow stranded-wire body and, as a result, the diameter of the outer circumference of the hollow stranded-wire body gradually decreases from the rear end portion side toward the tip portion side.

The hollow stranded-wire body of the guidewire according to the present invention may include a large-diameter unit and a small-diameter unit having a smaller strand diameter than the large-diameter unit. The stepped portion is desirably formed by the connection between the large-diameter unit and the small-diameter unit.

The guidewire according to the present invention may also include a coiled body formed by winding a single strand. The coiled body desirably covers the tip portion of the hollow stranded-wire body as a result of the engagement between one end portion of the coiled body and the stepped portion.

### BRIEF DESCRIPTION OF DRAWINGS

The foregoing and other objects, features, aspects and advantages of the invention will become more apparent from the following detailed description when taken in conjunction with the accompanying drawings.

FIG. 1A is a schematic plan view illustrating a guidewire according to one embodiment of the present invention.

FIG. 1B is a partially cutaway enlarged sectional view along the longitudinal direction of a distal portion of the guidewire illustrated in FIG. 1A.

FIG. 2A is a schematic plan view illustrating a guidewire according to another embodiment of the present invention.

FIG. 2B is a partially cutaway enlarged sectional view along the longitudinal direction of a distal portion of the guidewire illustrated in FIG. 2A.

FIG. 3A is a schematic plan view illustrating a guidewire according to still another embodiment of the present invention.

FIG. 3B is a partially cutaway enlarged sectional view along the longitudinal direction of a distal portion of the guidewire illustrated in FIG. 3A.

### DESCRIPTION OF EMBODIMENTS

Preferred embodiments of the present invention are described below with reference to the accompanying drawings, in which like reference characters designate similar or identical parts throughout the several views thereof.

### First Embodiment

A guidewire according to a first embodiment of the present invention includes: a core shaft having a tip portion and a rear end portion; and a hollow stranded-wire body formed by twisting a plurality of strands, wherein the core shaft includes: a small-diameter portion including the tip portion and extending over a predetermined area from the tip portion toward the rear end portion; and a large-diameter portion located nearer to the rear end portion than the small-diameter portion and extending over a predetermined area in the direction from the tip portion toward the rear end portion, and the small-diameter portion is inserted into, and thus covered with, the hollow stranded-wire body which, as a result, partially constitutes an outer circumferential surface of the guidewire.

In the guidewire according to the first embodiment of the present invention with the above configuration, the distal portion of the guidewire has an excellent torque.

The configuration and advantages of this guidewire are described in detail below with reference to the drawings.

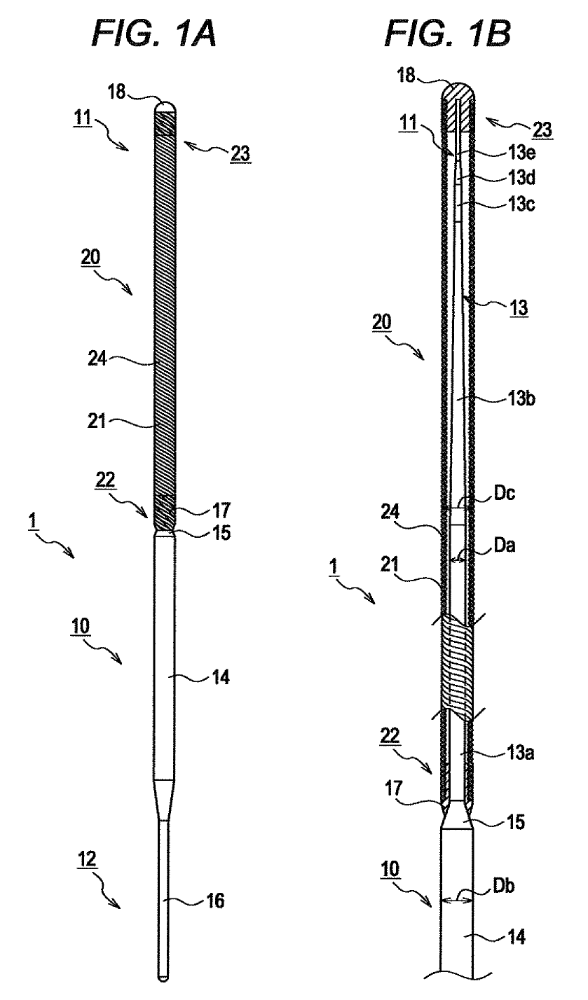

FIG. 1A is a schematic plan view illustrating a guidewire according to one embodiment of the present invention. FIG. 1B is a partially cutaway enlarged sectional view along the longitudinal direction of a distal portion of the guidewire illustrated in FIG. 1A.

A guidewire 1 illustrated in FIG. 1A includes a core shaft 10 and a hollow stranded-wire body 20. The core shaft 10 includes a tip portion 11 and a rear end portion 12. The hollow stranded-wire body 20 is formed by twisting a plurality of strands 21. To simplify the explanation, a distal portion of the guidewire 1 and the tip portion of the core shaft 10 are both denoted with the reference numeral 11 in FIG. 1A. Also in FIG. 1A, a proximal portion of the guidewire 1 and the rear end portion of the core shaft 10 are both denoted with the reference numeral 12.

More specifically, as illustrated in FIGS. 1A and 1B, the core shaft 10 includes a small-diameter portion 13 and a large-diameter portion 14. The small-diameter portion 13 includes the tip portion 11 and extends over a predetermined area from the tip portion 11 toward the rear end portion 12. The large-diameter portion 14 is located nearer to the rear end portion 12 than the small-diameter portion 13. In addition, the large-diameter portion 14 extends over a predetermined area in the direction from the tip portion 11 toward the rear end portion 12. In this manner, the diameter of the core shaft 10 decreases from the rear end portion 12 toward the tip portion 11. In other words, the core shaft 10 has a tapered shape. Therefore, the small-diameter portion 13 has a low stiffness and a high flexibility. The large-diameter portion 14, on the other hand, has a high stiffness and a low flexibility. The guidewire 1 including the core shaft 10 with the above configuration has the highly flexible distal portion 11, and can therefore reduce the damage to an inner wall of a vessel.

The small-diameter portion 13 of the core shaft 10 is inserted into the hollow stranded-wire body 20. In other words, the hollow stranded-wire body 20 covers the small-diameter portion 13. Thus, the hollow stranded-wire body 20 partially constitutes the outer circumferential surface of the guidewire 1.

The hollow stranded-wire body 20 is a tubular body formed by twisting the plurality of strands 21. This makes the hollow stranded-wire body 20 flexible. The hollow stranded-wire body 20 is less likely to be twisted despite the torsional force applied when a first end portion thereof is rotated. That is, torque generated at the first end portion can efficiently be transmitted to a second end portion. In this respect, the hollow stranded-wire body 20 is different from a coiled body formed by spirally winding a single strand (hereinafter simply referred to as “coiled body”).

The hollow stranded-wire body 20 with an excellent torque transmissibility is used, instead of a coiled body, in the guidewire 1. In the guidewire 1, therefore, torque generated at the proximal portion 12 by the rotation thereof is easily transmitted to the distal portion 11. This makes it easy for the distal portion of the guidewire 1 to pass through even a lesion site which is hardened or includes a severe stenosis or an occlusion. Note that, in general, the core shaft 10 has a higher stiffness than the hollow stranded-wire body 20. Therefore, the effect of the decrease in stiffness of the distal portion 11 of the guidewire 1 caused by the small-diameter portion 13 of the core shaft 10 is greater than that of the increase in stiffness of the distal portion 11 caused by the hollow stranded-wire body 20. This makes it unlikely to reduce the flexibility of the distal portion 11 of the guidewire 1 so severely as to damage the inner wall of a vessel, even by using the hollow stranded-wire body 20 instead of a coiled body. Note that the expression “the hollow stranded-wire body partially constitutes the outer circumferential surface of the guidewire” used herein means that the outer circumferential surface of the hollow stranded-wire body is part of the outer circumferential surface of the guidewire. Therefore, the hollow stranded-wire body is exposed as part of the outer circumferential surface of the guidewire and externally visible.

The guidewire according to the first embodiment of the present invention is described in more detail below with reference to the drawings.

The small-diameter portion 13 includes a first cylindrical portion 13a, a first tapered portion 13b, a second cylindrical portion 13c, a second tapered portion 13d, and a most distal portion 13e. The diameter of the first cylindrical portion 13a is substantially equal to the maximum diameter Da of the small-diameter portion 13. The first tapered portion 13b is connected to the first cylindrical portion 13a. The diameter of the first tapered portion 13b decreases toward the tip portion 11. The second cylindrical portion 13c is connected to the first tapered portion 13b. The second tapered portion 13d is connected to the second cylindrical portion 13c. The diameter of the second tapered portion 13d decreases toward the tip portion 11. The most distal portion 13e is connected to the second tapered portion 13d.

The most distal portion 13e has a rectangular cross-section in the direction perpendicular to the longitudinal direction of the core shaft 10. More specifically, this cross-section has an elongated rectangular shape. In contrast, the cross-sectional shape of the second tapered portion 13d in the above direction gradually transforms from circle into rectangle toward the most distal portion 13e from the second cylindrical portion 13c.

The large-diameter portion 14 has a cylindrical shape, and has the maximum diameter Db larger than the maximum diameter Da of the small-diameter portion 13. The first cylindrical portion 13a is coupled to the large-diameter portion 14 via a tapered coupling portion 15. A connecting portion 16 for connecting an extension guidewire (not shown) is attached to an end portion of the large-diameter portion 14 on the side of the rear end portion 12 of the core shaft 10.

The hollow stranded-wire body 20 is a tubular member having a through hole extending in the axial direction. The hollow stranded-wire body 20 is formed by twisting the plurality of strands 21. More specifically, the hollow stranded-wire body 20 is formed by spirally twisting the plurality of strands 21 having a circular cross-section. Therefore, a plurality of spiral grooves 24 are formed on the outer circumferential surface of the hollow stranded-wire body 20. Each groove 24 is formed between one strand 21 and another strand 21 next to the one strand 21.

The small-diameter portion 13 is inserted into (the through hole of) the hollow stranded-wire body 20. In other words, the small-diameter portion 13 is covered with the hollow stranded-wire body 20. In this manner, the outer circumferential surface of the hollow stranded-wire body 20 partially constitutes the outer circumferential surface of the guidewire 1. That is, the outer circumferential surface of the guidewire 1 includes the outer circumferential surface of the hollow stranded-wire body 20 and the outer circumferential surface of the large-diameter portion 14 of the core shaft 10.

The first cylindrical portion 13a of the small-diameter portion 13 and a rear end portion 22 of the hollow stranded-wire body 20 are fixed to each other via a rear-end brazing portion 17. The most distal portion 13e of the small-diameter portion 13 and a tip portion 23 of the hollow stranded-wire body 20 are fixed to each other via a semispherical front-end brazing portion 18.

The maximum diameter Db of the large-diameter portion 14 is substantially equal to the maximum diameter Dc of the hollow stranded-wire body 20. Herein, the fact that the maximum diameter of the large-diameter portion is substantially equal to the maximum diameter of the hollow stranded-wire body means that the large-diameter portion has the same maximum diameter as the hollow stranded-wire body, or that the difference, if any, between the maximum diameters is so small that resistance is hardly generated upon insertion of the guidewire into the body.

The guidewire of the present embodiment can be produced, for example, by a production method including the steps of: forming a core shaft having the predetermined shape described above by tapering a metal rod material; inserting a small-diameter portion of the formed core shaft into a hollow stranded-wire body; and brazing the small-diameter portion with the hollow stranded-wire body at a predetermined position. Examples of the tapering process include a cutting process such as centering grinding, a swaging process, and a drawing process.

Effects of the guidewire of the present embodiment are listed below.

(1) In the guidewire of the present embodiment, the small-diameter portion of the core shaft is covered with the hollow stranded-wire body formed by twisting a plurality of strands and excellent in torque transmissibility. The hollow stranded-wire body partially constitutes the outer circumferential surface of the guidewire. Thus, rotating the proximal portion of the guidewire allows torque generated at the proximal portion to be easily transmitted to the distal portion. Therefore, the guidewire of the present embodiment has an excellent torque transmissibility, so that the distal portion of the guidewire can easily pass through a lesion site.

(2) In the guidewire of the present embodiment, the large-diameter portion has substantially the same maximum diameter as the hollow stranded-wire body. The guidewire is substantially cylindrical in shape with a constant diameter, and the outer circumferential surface of the guidewire is smoother, so that the distal portion of the guidewire can more easily pass through the lesion site.

(3) The small-diameter portion includes the first cylindrical portion, the first tapered portion, the second cylindrical portion, the second tapered portion, and the most distal portion. The diameter of the small-diameter portion gradually decreases toward the tip portion thereof. Therefore, the stiffness of the small-diameter portion gradually decreases toward the tip portion thereof. Along with the decrease in stiffness, on the other hand, the flexibility of the small-diameter portion gradually increases. The small-diameter portion does not include a part where the diameter abruptly decreases. Therefore, an external bending force is less likely to concentrate on one point, and thus the small-diameter portion is hardly subject to plastic deformation.

(4) The most distal portion of the core shaft has a rectangular cross-section along the direction perpendicular to the longitudinal direction. This most distal portion has a higher flexibility than a most distal portion with a circular cross-section.

(5) The second tapered portion of the small-diameter portion has a cross-sectional shape in the direction perpendicular to the longitudinal direction. Such a cross-section gradually transforms from circle into rectangle toward the most distal portion from the second cylindrical portion. In other words, this cross-sectional shape does not change abruptly. Therefore, an external bending force is less likely to concentrate on the small-diameter portion. As a result, plastic deformation hardly occurs around the tip portion of the core shaft.

(6) A plurality of spiral grooves are formed on the outer circumferential surface of the hollow stranded-wire body. Therefore, the hollow stranded-wire body has an external-thread shape. Thus, the distal portion of the guidewire is screwed into the lesion site by the rotation of the proximal portion of the guidewire. Therefore, the guidewire of the present embodiment has an excellent crossability to a lesion site.

### Second Embodiment

A guidewire according to a second embodiment of the present invention is described below with reference to the drawings. The guidewire of the present embodiment has the same configuration as the guidewire of the first embodiment described above, except that a stepped portion is formed on the outer circumferential surface of the hollow stranded-wire body and, as a result, the diameter of the outer circumference of the hollow stranded-wire body gradually decreases from a rear end portion toward a tip portion. More specifically, the hollow stranded-wire body includes a large-diameter unit and a small-diameter unit having a smaller strand diameter than the large-diameter unit. The large-diameter unit and the small-diameter unit are connected to each other. Thus, the stepped portion is formed. The guidewire of the present embodiment has the same configuration as the guidewire of the first embodiment described above except this configuration. Therefore, overlapping features with the guidewire of the first embodiment will not be repeated.

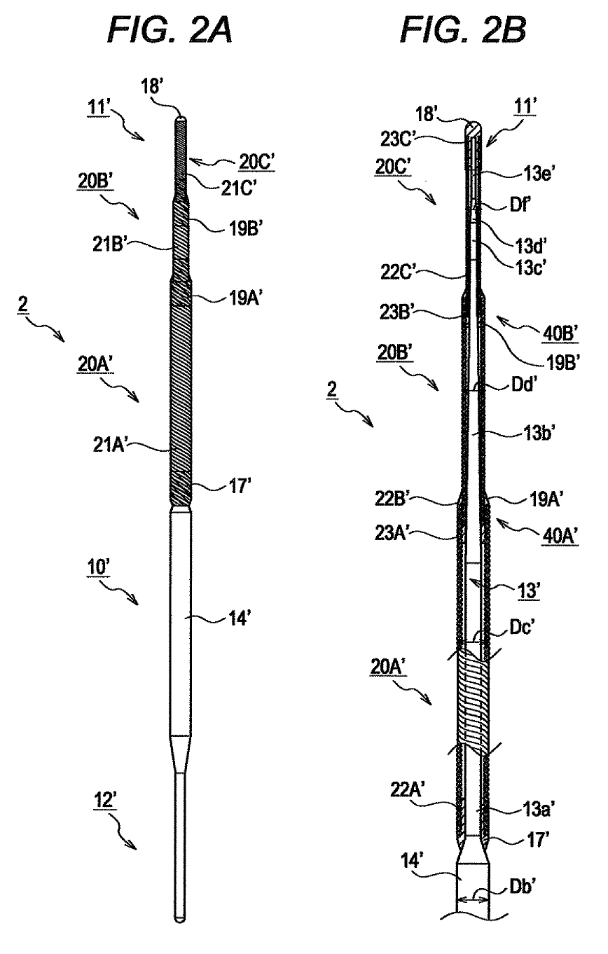

FIG. 2A is a plan view schematically illustrating a guidewire according to the present embodiment. FIG. 2B is a partially cutaway enlarged sectional view along the longitudinal direction of a distal portion of the guidewire illustrated in FIG. 2A.

A guidewire 2 of the present embodiment illustrated in FIGS. 2A and 2B includes a core shaft 10′ having a tip portion 11′ and a rear end portion 12′, a first hollow stranded-wire body 20A′ formed by twisting a plurality of strands 21A′, a second hollow stranded-wire body 20B′ formed by twisting a plurality of strands 21B′, and a third hollow stranded-wire body 20C′ formed by twisting a plurality of strands 21C′.

The configuration of the core shaft 10′ is similar to that of the core shaft of the guidewire described in the above first embodiment.

The first hollow stranded-wire body 20A′ covers both an entire first cylindrical portion 13a′ of a small-diameter portion 13′ and a rear end portion of a first tapered portion 13b′. Note that, in FIG. 2B, the maximum diameter (outer diameter) of the first hollow stranded-wire body 20A′ is denoted with the reference symbol Dc′.

The maximum diameter Dd′ (outer diameter) of the second hollow stranded-wire body 20B′ is substantially equal to the inner diameter of the first hollow stranded-wire body 20A′, but smaller than the maximum diameter Dc′ of the first hollow stranded-wire body 20A′. Regarding the relationship between the first hollow stranded-wire body 20A′ and the second hollow stranded-wire body 20B′, therefore, the first hollow stranded-wire body 20A′ corresponds to the large-diameter unit, and the second hollow stranded-wire body 20B′ corresponds to the small-diameter unit. The strands constituting the second hollow stranded-wire body 20B′ have a smaller diameter than the strands constituting the first hollow stranded-wire body 20A′. Therefore, the second hollow stranded-wire body 20B′ has a smaller outer diameter than the first hollow stranded-wire body 20A′ but has a sufficiently large inner diameter. The second hollow stranded-wire body 20B′ covers an area from the rear end portion to the tip portion of the first tapered portion 13b′.

A first joint 40A′ is provided for connecting a rear end portion 22B′ of the second hollow stranded-wire body 20B′ to a tip portion 23A′ of the first hollow stranded-wire body 20A′. The rear end portion 22B′ of the second hollow stranded-wire body 20B′ is fitted into the tip portion 23A′ of the first hollow stranded-wire body 20A′ so as to come into contact with an inner wall of the tip portion 23A′ of the first hollow stranded-wire body 20A′. As a result, a stepped portion is formed on the first joint 40A′. Thus, the diameter of the outer circumference of the hollow stranded-wire body gradually decreases from the rear end portion 12′ toward the tip portion 11′. In this manner, the stepped portion is formed by the connection between the large-diameter unit (first hollow stranded-wire body 20A′) and the small-diameter unit (second hollow stranded-wire body 20B′).

The maximum diameter Df′ (outer diameter) of the third hollow stranded-wire body 20C′ is substantially equal to the inner diameter of the second hollow stranded-wire body 20B′, and smaller than the maximum diameter Dd′ of the second hollow stranded-wire body 20B′. Regarding the relationship between the second hollow stranded-wire body 20B′ and the third hollow stranded-wire body 20C′, therefore, the second hollow stranded-wire body 20B′ corresponds to the large-diameter unit, and the third hollow stranded-wire body 20C′ corresponds to the small-diameter unit. The strands constituting the third hollow stranded-wire body 20C′ have a smaller diameter than the strands constituting the second hollow stranded-wire body 20B′. Therefore, the third hollow stranded-wire body 20C′ has a smaller outer diameter than the second hollow stranded-wire body 20B′ but has a sufficiently large inner diameter. The third hollow stranded-wire body 20C′ covers the tip portion of the first tapered portion 13b′, a second cylindrical portion 13c′, a second tapered portion 13d′, and a most distal portion 13e′.

A second joint 40B′ is provided for connecting a rear end portion 22C′ of the third hollow stranded-wire body 20C′ to a tip portion 23B′ of the second hollow stranded-wire body 20B′. The rear end portion 22C′ of the third hollow stranded-wire body 20C′ is fitted into the tip portion 23B′ of the second hollow stranded-wire body 20B′ so as to come into contact with an inner wall of the tip portion 23B′ of the second hollow stranded-wire body 20B′. As a result, a stepped portion is formed on the second joint 40B′. Thus, the diameter of the outer circumference of the hollow stranded-wire body gradually decreases from the rear end portion 12′ toward the tip portion 11′. In this manner, the stepped portion is formed by the connection between the large-diameter unit (second hollow stranded-wire body 20B′) and the small-diameter unit (third hollow stranded-wire body 20C′).

As described above, the small-diameter portion 13′ of the core shaft 10′ is inserted into the first hollow stranded-wire body 20A′, the second hollow stranded-wire body 20B′, and the third hollow stranded-wire body 20C′. In other words, the small-diameter portion 13′ is covered with the first hollow stranded-wire body 20A′, the second hollow stranded-wire body 20B′, and the third hollow stranded-wire body 20C′. Thus, the first hollow stranded-wire body 20A′, the second hollow stranded-wire body 20B′, and the third hollow stranded-wire body 20C′ partially constitute the outer circumferential surface of the guidewire 2.

The maximum diameter Db′ of the large-diameter portion 14′ is substantially equal to the maximum diameter Dc′ of the first hollow stranded-wire body 20A′.

A rear end portion 22A′ of the first hollow stranded-wire body 20A′ and the rear end portion of the first cylindrical portion 13a′ of the small-diameter portion 13′ are fixed to each other via a rear-end brazing portion 17′. At the first joint 40A′, the tip portion 23A′ of the first hollow stranded-wire body 20A′, the rear end portion 22B′ of the second hollow stranded-wire body 20B′, and the rear end portion of the first tapered portion 13b′ are fixed to one another via a first middle brazing portion 19A′. At the second joint 40B′, the tip portion 23B′ of the second hollow stranded-wire body 20B′, the rear end portion 22C′ of the third hollow stranded-wire body 20C′, and the tip portion of the first tapered portion 13b′ are fixed to one another via a second middle brazing portion 19B′. A tip portion 23C′ of the third hollow stranded-wire body 20C′ and the most distal portion 13e′ of the small-diameter portion 13′ are fixed to each other via a semispherical front-end brazing portion 18′.

Effects of the guidewire of the present embodiment are listed below. The guidewire of the present embodiment has the effects (1) to (6) of the guidewire of the first embodiment described above.

In addition, the guidewire of the present embodiment has unique effects (7) and (8) listed below.

(7) The stepped portion is formed on the outer circumferential surface of the hollow stranded-wire body. Thus, the diameter of the outer circumference of the hollow stranded-wire body gradually decreases from the rear end portion toward the tip portion. Therefore, the guidewire is gradually tapered toward the distal portion thereof. As a result, the guidewire of the present embodiment has a better crossability to a lesion site.

(8) The hollow stranded-wire body has the large-diameter unit and the small-diameter unit having a smaller strand diameter than the large-diameter unit. Furthermore, the stepped portion is formed at the connecting portion between the large-diameter unit and the small-diameter unit. In this manner, a plurality of units having various outer diameters and inner diameters can be connected to each other. Thus, the shape of the stepped portion formed on the outer circumferential surface of the hollow stranded-wire body can be freely designed.

### Third Embodiment

A guidewire according to a third embodiment of the present invention is described below with reference to the drawings. The guidewire of the present embodiment has the same configuration as the guidewire of the first embodiment described above, except that the former guidewire further includes a coiled body formed by winding a single strand and that the coiled body covers a tip portion of a hollow stranded-wire body. Therefore, overlapping features will not be repeated.

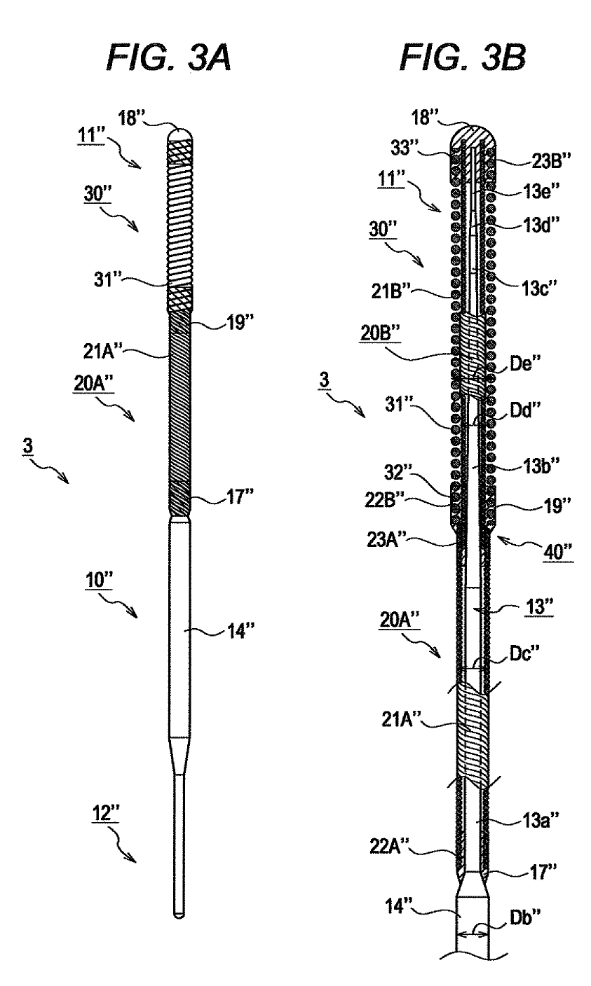

FIG. 3A is a plan view schematically illustrating a guidewire according to the present embodiment. FIG. 3B is a partially cutaway enlarged sectional view along the longitudinal direction of a distal portion of the guidewire illustrated in FIG. 3A.

A guidewire 3 of the present embodiment illustrated in FIGS. 3A and 3B includes a core shaft 10″ having a tip portion 11″ and a rear end portion 12″, a first hollow stranded-wire body 20A″ formed by twisting a plurality of strands 21A″, a second hollow stranded-wire body 20B″ formed by twisting a plurality of strands 21B″, and a coiled body 30″ formed by winding a single strand 31″.

The configuration of the core shaft 10″ is similar to that of the core shaft of the guidewire of the first embodiment described above.

The first hollow stranded-wire body 20A″ covers an entire first cylindrical portion 13a″ of a small-diameter portion 13″, and a rear end portion of a first tapered portion 13b″. Note that, in FIG. 3B, the maximum diameter (outer diameter) of the first hollow stranded-wire body 20A″ is denoted with the reference symbol Dc″.

The maximum diameter Dd″ (outer diameter) of the second hollow stranded-wire body 20B″ is substantially equal to the inner diameter of the first hollow stranded-wire body 20A″ and smaller than the maximum diameter Dc″ of the first hollow stranded-wire body 20A″. Regarding the relationship between the first hollow stranded-wire body 20A″ and the second hollow stranded-wire body 20B″, therefore, the first hollow stranded-wire body 20A″ corresponds to the large-diameter unit and the second hollow stranded-wire body 20B″ corresponds to the small-diameter unit. The strands constituting the second hollow stranded-wire body 20B″ have a smaller diameter than the strands constituting the first hollow stranded-wire body 20A″. Therefore, the second hollow stranded-wire body 20B′ has a smaller outer diameter than the first hollow stranded-wire body 20A″, while having a sufficiently large inner diameter. The second hollow stranded-wire body 20B″ covers the rear end portion of the first tapered portion 13b″, and the rest of the small-diameter portion 13″ on the side of the tip portion 11″ from the rear end portion of the first tapered portion 13b″. In other words, the second hollow stranded-wire body 20B″ covers the rear end portion of the first tapered portion 13b″, a second cylindrical portion 13c″, a second tapered portion 13d″, and a most distal portion 13e″.

A joint 40″ is for connecting a rear end portion 22B″ of the second hollow stranded-wire body 20B″ to a tip portion 23A″ of the first hollow stranded-wire body 20A″. The rear end portion 22B″ of the second hollow stranded-wire body 20B″ is fitted into the tip portion 23A″ of the first hollow stranded-wire body 20A″ so as to come into contact with an inner wall of the tip portion 23A″ of the first hollow stranded-wire body 20A″. As a result, a stepped portion is formed on the joint 40″. Thus, the diameter of the hollow stranded-wire body decreases from the rear end portion 12″ toward the tip portion 11″. In this manner, the stepped portion is formed by the connection between the large-diameter unit (first hollow stranded-wire body 20A″) and the small-diameter unit (second hollow stranded-wire body 20B′).

The small-diameter portion 13″ of the core shaft 10″ is inserted into the first hollow stranded-wire body 20A″ and the second hollow stranded-wire body 20B″. In other words, the small-diameter portion 13″ is covered with the first hollow stranded-wire body 20A″ and the second hollow stranded-wire body 20B″. Thus, the first hollow stranded-wire body 20A″ and the second hollow stranded-wire body 20B″ partially constitute the outer circumferential surface of the guidewire 3.

The maximum diameter Db″ of the large-diameter portion 14″ is substantially equal to the maximum diameter Dc″ of the first hollow stranded-wire body 20A″.

The inner diameter of the coiled body 30″ is substantially equal to the inner diameter of the first hollow stranded-wire body 20A″ and the maximum diameter Dd″ (outer diameter) of the second hollow stranded-wire body 20B″. The maximum diameter De″ (outer diameter) of the coiled body 30″ is larger than the maximum diameter Dc″ (outer diameter) of the first hollow stranded-wire body 20A″. In addition, the coiled body 30″ is arranged so that a rear end portion 32″ thereof can be engaged with the stepped portion of the joint 40″. A tip portion 33″ of the coiled body 30″ reaches the most distal portion 13e″. Thus, the coiled body 30″ covers the second hollow stranded-wire body 20B″.

In this manner, the coiled body 30″ covers the second hollow stranded-wire body 20B″ that is the tip portion of the hollow stranded-wire body (first hollow stranded-wire body 20A″ and second hollow stranded-wire body 20B″).

A rear end portion 22A″ of the first hollow stranded-wire body 20A″ and a rear end portion of the first cylindrical portion 13a″ of the small-diameter portion 13″ are fixed to each other via a rear-end brazing portion 17″. At the joint 40″, the tip portion 23A″ of the first hollow stranded-wire body 20A″, the rear end portion 22B″ of the second hollow stranded-wire body 20B″, the rear end portion 32″ of the coiled body 30″, and the rear end portion of the first tapered portion 13b″ are fixed to one another via a middle brazing portion 19″. The tip portion 23B″ of the second hollow stranded-wire body 20B″, the tip portion 33″ of the coiled body 30″, and the most distal portion 13e″ of the small-diameter portion 13″ are fixed to one another via a semispherical front-end brazing portion 18″.

Effects of the guidewire of the present embodiment are listed below. The guidewire of the present embodiment has the effects (1) to (6) of the guidewire of the first embodiment described above.

In addition, the guidewire of the present embodiment has unique effects (9) and (10) listed below.

(9) The coiled body formed by winding a strand covers the tip portion of the hollow stranded-wire body. Therefore, by appropriately changing the material for the strand of the coiled body, the guidewire can have desired features such as radiopaque properties.

(10) The rear end portion of the coiled body is engaged with the stepped portion of the joint. This reduces the maximum diameter (outer diameter) of the guidewire. As a result, the guidewire of the present embodiment has an excellent crossability to a lesion site. In addition, the coiled body is less likely to come off during an operation. Therefore, this guidewire is highly safe.

### Other Embodiments

In the guidewire according to the present invention, desirably, the large-diameter portion has substantially the same maximum diameter as the hollow stranded-wire body. Alternatively, however, the large-diameter portion may have a larger maximum diameter than the hollow stranded-wire body. Further alternatively, the large-diameter portion may have a smaller maximum diameter than the hollow stranded-wire body. Even in the latter two cases, the advantageous effects of the present invention can be favorably obtained.

Preferred examples of the material for the strand constituting the hollow stranded-wire body of the guidewire according to the present invention include: stainless steel such as martensite-based stainless steel, ferrite-based stainless steel, austenite-based stainless steel, austenite, ferrite duplex stainless steel, and precipitation-hardened stainless steel; super elastic alloys such as an Ni—Ti alloy; and tungsten.

In the guidewire according to the present invention, the stepped portion may be formed on the outer circumferential surface of the hollow stranded-wire body so that the diameter of the outer circumference of the hollow stranded-wire body gradually decreases from the rear end portion toward the tip portion. As described above, the stepped portion may be formed by the connection between the large-diameter unit and the small-diameter unit. Alternatively, when the hollow stranded-wire body consists of one seamless unit without being divided, the stepped portion may be formed on the outer circumferential surface of that unit.

When the hollow stranded-wire body includes the large-diameter unit and the small-diameter unit, the number of units is not limited to two or three as described, but may be four or more. The more units, the smaller the stepped portion on the joint between the units. This further reduces the resistance caused upon insertion of the guidewire into the body. As a result, the distal portion of the guidewire has a particularly good crossability to a lesion site.

Preferred examples of the material for the strand constituting the coiled body of the guidewire according to the present invention include: stainless steel such as martensite-based stainless steel, ferrite-based stainless steel, austenite-based stainless steel, austenite, ferrite duplex stainless steel, and precipitation-hardened stainless steel; super elastic alloys such as an Ni—Ti alloy; and radiopaque metals such as platinum, gold, and tungsten.

Preferred examples of the material for the core shaft of the guidewire according to the present invention include: stainless steel; super elastic alloys such as an Ni—Ti alloy; a piano wire; and a tungsten wire. Examples of the stainless steel include martensite-based stainless steel, ferrite-based stainless steel, austenite-based stainless steel, austenite, ferrite duplex stainless steel, and precipitation-hardened stainless steel. Among these, the austenite-based stainless steel is desirable, and particularly desirable examples thereof include SUS304, SUS316, and SUS316L.

Examples of the material for brazing metal forming the brazing portion of the guidewire according to the present invention include aluminum alloy solder, silver solder, gold solder, zinc, Sn—Pb alloy, Sn—Au alloy, Pb—Ag alloy, and Sn—Ag alloy. Among these, gold solder, Sn—Au alloy, and Sn—Ag alloy are particularly preferable from the viewpoint of further increasing the strength of the brazing portion.

The outer surface of the guidewire according to the present invention may be coated with a hydrophilic material. Such coating makes it possible to reduce the sliding resistance of the guidewire inside a guiding catheter, a tubular organ, or an intracorporeal tissue. As a result, the guidewire can be moved smoothly.

Examples of the hydrophilic material include cellulose-based polymeric substance, polyethylene oxide-based polymeric substance, maleic anhydride-based polymeric substance (e.g., maleic anhydride copolymer such as methylvinyl ether-maleic anhydride copolymer), acrylamide-based polymeric substance (e.g., polyacrylamide, and polyglycidyl methacrylate-dimethylacrylamide block copolymer), water-soluble nylon, polyvinyl alcohol, polyvinylpyrrolidone, and hyaluronate. Among these, hyaluronate is desirable.

While the invention has been shown and described in detail, the foregoing description is in all aspects illustrative and not restrictive. It is therefore understood that numerous modifications and variations can be devised without departing from the spirit and scope of the invention.

### REFERENCE SIGNS LIST

1, 2, 3 guidewire
10, 10′, 10″ core shaft
11, 11′, 11″ tip portion of core shaft
12, 12′, 12″ rear end portion of core shaft
13, 13′, 13″ small-diameter portion of core shaft
14, 14′, 14″ large-diameter portion of core shaft
20, 20A′, 20B′, 20C′, 20A″, 20B″ hollow stranded-wire body
21, 21A′, 21B′, 21C′, 21A″, 21B″ strand of hollow stranded-wire body

1. A guidewire comprising:
a core shaft having a tip portion and a rear end portion; and
a hollow stranded-wire body formed by twisting a plurality of strands,
wherein the core shaft comprises:
a small-diameter portion comprising the tip portion and extending over a predetermined area from the tip portion toward the rear end portion; and
a large-diameter portion located nearer to the rear end portion than the small-diameter portion and extending over a predetermined area in a direction from the tip portion toward the rear end portion, and
the small-diameter portion is inserted into the hollow stranded-wire body and covered with the hollow stranded-wire body to partially constitute an outer circumferential surface of the guide wire.

2. The guidewire according to claim 1, wherein the large-diameter portion has substantially the same maximum diameter as the hollow stranded-wire body.

3. The guidewire according to claim 1, wherein a stepped portion is formed on an outer circumferential surface of the hollow stranded-wire body and, as a result, a diameter of an outer circumference of the hollow stranded-wire body gradually decreases from the rear end portion side toward the tip portion side.

4. The guidewire according to claim 3, wherein the hollow stranded-wire body comprises a large-diameter unit and a small-diameter unit having a smaller strand diameter than the large-diameter unit, and
the stepped portion is formed by connection between the large-diameter unit and the small-diameter unit.

5. The guidewire according to claim 3, further comprising:
a coiled body formed by winding a single strand,
wherein the coiled body covers the tip portion of the hollow stranded-wire body by engagement between one end portion of the coiled body and the stepped portion.
